(19) United States
(12) Patent Application Publication
(10) Pub. No.: US 2024/0398787 A1
(43) Pub. Date: Dec. 5, 2024

(54) TREATMENT OF MAJOR DEPRESSIVE DISORDER

(71) Applicants: AbbVie Inc., North Chicago, IL (US); Allergan Pharmaceuticals International Limited, Dublin (IE)

(72) Inventors: Kumar Budur, Chicago, IL (US); Ludmyla Rekeda, Hoboken, NJ (US); Willie R. Earley, West Chester, PA (US)

(21) Appl. No.: 18/437,732

(22) Filed: Feb. 9, 2024

Related U.S. Application Data
(62) Division of application No. 18/050,907, filed on Oct. 28, 2022, now abandoned.
(60) Provisional application No. 63/263,213, filed on Oct. 28, 2021.
(60) Provisional application No. 63/362,446, filed on Apr. 4, 2022.

(51) Int. Cl. A61K 31/495; A61P 25/24

(52) U.S. Cl. CPC A61K 31/495; A61P 25/24

(57) ABSTRACT

The present disclosure provides methods for the treatment of major depressive disorder by administering cariprazine or a pharmaceutically acceptable salt thereof.

### CROSS-REFERENCE TO RELATED APPLICATIONS

The present application claims the benefit of U.S. Provisional Application No. 63/263,213 filed Oct. 28, 2021, and U.S. Provisional Application No. 63/362,446, filed Apr. 4, 2022, the content of each of which is incorporated by reference herein in its entirety.

### FIELD

The present disclosure is related to medicaments and methods for treating major depressive disorder, and more specifically as adjunctive treatment to antidepressant therapy for major depressive disorder.

### BACKGROUND

Treatment of major depressive disorder (MDD) is complex and often involves psychotherapy, education and use of various pharmacotherapeutic agents. Antidepressants (ADTs) are the primary pharmacotherapeutic agents used in the treatment of patients suffering from MDD. Currently, ADTs such as aripiprazole, quetiapine fumarate, and brexipiprazole, are approved for treatment of patients suffering from MDD. Despite the number of available treatment options, many patients fail to achieve adequate response or remission of their anxiety symptoms with monotherapy use of ADTs alone. More than half of patients never experience satisfactory results. There remains a need for optimized and targeted methodologies and dosing regimens to adequately treat major depressive disorder.

It is often unclear whether a patient's response to an MDD treatment is due to the treatment or a placebo effect. A 2-point or more change in the Montgomery Åsberg Depression Rating Scale (MADRS) total score between treatment and placebo has been commonly used to indicate a clinically relevant treatment effect. However, without conducting human clinical trials, it has been almost impossible to predict whether a particular treatment regimen would achieve such clinical relevancy. Durgam et al., J Clin. Psychiatry 77:371-378 (2016), which is incorporated herein by reference in its entirety, describes a randomized double-blinded, placebo-controlled flexible-dose study testing whether cariprazine could be used as an adjunctive treatment to treat MDD patients who had inadequate response to ongoing antidepressant treatment. The study showed that cariprazine 1-2 mg per day variable dosing did not achieve statistically significant difference in the MADRS total score as compared to placebo (the least squares mean difference (LSMD)=−0.9, adjusted P value=0.2404), while 2-4.5 mg per day variable dosing did (LSMD=−2.2, adjusted P value=0.0114).

### SUMMARY

The present invention unexpectedly discovered that cariprazine 1.5 mg per day as an adjunctive treatment for MDD patients achieved a statistically significant change in the MADRS total score compared to placebo (LSMD=−2.5, Adjusted P value=0.005) and therefore is a clinically relevant treatment for MDD.

The present disclosure provides methods of treating major depressive disorder, involving administering cariprazine or a pharmaceutically acceptable salt thereof as an adjunct to antidepressant therapy in the treatment of subjects with major depressive disorder (MDD).

In embodiments, the present disclosure provides methods of treating major depressive disorder, involving administering cariprazine or a pharmaceutically acceptable salt thereof in an amount of 1.5 mg/day in addition to ADT therapy or 3 mg/day in addition to ADT therapy, to a patient in need of treatment thereof.

In embodiments, the present disclosure provides methods of treating major depressive disorder, involving administering cariprazine or a pharmaceutically acceptable salt thereof in an amount of 1.5 mg/day in addition to ADT therapy or 3 mg/day in addition to ADT therapy to a patient in need of treatment thereof for 6 weeks.

In embodiments, the present disclosure provides methods of treating major depressive disorder comprising administering cariprazine or a pharmaceutically acceptable salt thereof in an amount of 1.5 mg/day or 3 mg/day to a patient in need of adjunctive therapy for major depressive disorder (MDD), wherein the administration of cariprazine achieves statistically significant change from baseline to Week 6 in the Montgomery Åsberg Depression Rating Scale (MADRS) total score.

In embodiments, a method comprises administering cariprazine or a pharmaceutically acceptable salt thereof in an amount of 1.5 mg/day or 3 mg/day to a patient in need of adjunctive therapy for MDD that meets Diagnostic and Statistical Manual of Mental Disorders, Fifth Edition (DSM-5) criteria for MDD based on Structured Clinical Interview for DSM-5 (SCID-5) and has a total score ≥22 on the Hamilton Depression Rating Scale-17 (HAMD-17) prior to treatment. In embodiments, the patient has a major depressive episode of at least 8 weeks to less than 24 months. In embodiments, the patient demonstrates inadequate response to 1 to 3 antidepressants of adequate dose and adequate duration as measured by Antidepressant Treatment Response Questionnaire (ATRQ).

In embodiments, a method of treating major depressive disorder comprises administering cariprazine or a pharmaceutically acceptable salt thereof in an amount of 1.5 mg/day as an adjunct to antidepressant therapy to a patient in need of treatment for major depressive disorder for 6 weeks. In embodiments, the administration of cariprazine or a pharmaceutically acceptable salt results in a statistically significant remission of the depressive episode as defined by less than 10 total score on MADRS total score. In embodiments, the statistically significant remission is achieved with less than 2% to no adverse events leading to discontinuation of therapy. In embodiments, cariprazine or a pharmaceutically acceptable salt is administered as cariprazine hydrochloride.

In embodiments, a method of treating major depressive disorder comprises administering cariprazine or a pharmaceutically acceptable salt thereof in an amount of 3.0 mg/day as an adjunct to antidepressant therapy to a patient in need of treatment for major depressive disorder for 6 weeks. In embodiments, the administration of cariprazine or a pharmaceutically acceptable salt results in a statistically significant remission of the depressive episode as defined by less than 10 total score on MADRS total score. In embodiments, the statistically significant remission is achieved with less than 2% to no adverse events leading to discontinuation of therapy. In embodiments, cariprazine or a pharmaceutically acceptable salt is administered as cariprazine hydrochloride.

In embodiments, the present disclosure provides a method of statistically significant treatment of major depressive disorder in patients who had inadequate responses to one or more antidepressants, comprising administering to each said patient cariprazine or a pharmaceutically acceptable salt thereof in an amount of 1.5 mg per day as an adjunctive therapy to one or more said antidepressants, and said treatment achieves statistically significant least square mean change in the MADRS total score in said patients from baseline to week 6 of said treatment as compared to placebo.

In embodiments, the method of statistically significant treatment of major depressive disorder in patients who had inadequate responses to one or more antidepressants, comprises administering to each said patient cariprazine or a pharmaceutically acceptable salt thereof in an amount of 1.5 mg per day as an adjunctive therapy to one or more said antidepressants, and said treatment achieves statistically significant least square mean change in the MADRS total score in said patients from baseline to week 6 of said treatment as compared to placebo, wherein said statistically significant least square mean change in the MADRS total score in said patients from baseline to week 6 of said treatment as compared to placebo is −2 points or exceeds said 2-points.

### BRIEF DESCRIPTION OF THE FIGURES

FIG. 1 shows a schematic of a clinical study conducted for evaluating the safety and efficacy of cariprazine administered as capsules once a day as an adjunct to antidepressant therapy.

FIG. 2 shows the LS Mean Change in MADRS Total Score change from Baseline (+/−SE) over time results by Weeks.

FIG. 3 shows the LS Mean Change in CGI-S Score change from Baseline (+/−SE) over time results by Weeks.

FIG. 4 shows the full range of MADRS score improvement at end of week 6 (LOCF).

### DETAILED DESCRIPTION

The present disclosure provides methods for treating major depressive disorder in a patient in need thereof. In embodiments, the present disclosure provides methods for the treating patients suffering from major depressive disorder. In embodiments, the present disclosure provides methods for treating major depressive disorder comprising administering a therapeutically effective amount of cariprazine or a pharmaceutically acceptable salt thereof as an adjunct to antidepressant therapy (ADT). The structure of cariprazine is shown below:

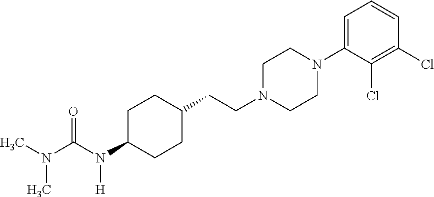

and has the chemical name of cariprazine, or 4-[[2-[4-(2,3-dichlorophenyl)piperazin-1-yl]ethyl]carbamoyl]cyclohexan-1yl]-N,N-dimethylamine or trans-4-[[2-[4-(2,3-dichlorophenyl)piperazin-1-yl]ethyl]carbamoyl]cyclohexan-1yl]-N,N-dimethylamine or trans-N-{4-[2-[4-(2,3-dichlorophenyl)piperazin-1-yl]ethyl]cyclohexyl}-N′,N′-dimethylurea. Cariprazine can be administered as the hydrochloride salt of cariprazine, that is 4-[[2-[4-(2,3-dichlorophenyl)piperazin-1-yl]ethyl]carbamoyl]cyclohexan-1yl]-N,N-dimethylamine hydrochloride or trans-4-[[2-[4-(2,3-dichlorophenyl)piperazin-1-yl]ethyl]carbamoyl]cyclohexan-1yl]-N,N-dimethylamine hydrochloride or trans-N-{4-[2-[4-(2,3-dichlorophenyl)piperazin-1-yl]ethyl]cyclohexyl}-N′,N′-dimethylurea hydrochloride. Preparation of cariprazine is described, for example, in U.S. Pat. No. 7,737,142, filed Jan. 20, 2006, and related foreign counterpart patents.

Treatment can reduce the frequency and intensity of symptoms associated with major depressive disorder. In embodiments, the methods can result in freedom from symptoms associated with major depressive disorder. In embodiments, the administration of cariprazine may provide for fewer symptoms or symptoms of reduced intensity. In embodiments, the symptoms of major depressive disorder may be reduced or eliminated.

An evaluation of symptom criteria for Major Depressive Episode/Disorder is outlined in “The Diagnostic and Statistical Manual of Mental Disorders,” 5^{th} edition (also known as the DSM-5). Factors may include: depressed mood; loss of interest in almost all activities; unintentional weight loss/gain or decrease/increase in appetite; sleep disturbances; psychomotor changes (e.g., agitation or retardation) severe enough to be observable by others; tiredness, fatigue or low energy, or decreased efficiency with which routine tasks are completed; a sense of worthlessness or excessive, inappropriate or delusional guilt; impaired ability to think concentrate, or make decisions; and recurrent thoughts of death, suicidal ideation or suicide attempts. The symptoms cause clinically significant distress or impairment in social, occupational, or other important areas of functioning. The symptoms are not due to the direct physiological effects of a substance (e.g., drug abuse, a prescribed medication's side effects) or a medication condition (e.g. hypothyroidism). A patient suffering from MDD has never had a manic episode or hypomanic episode. Their condition is not better explained by schizophrenia spectrum or other psychotic disorders. A patient in need of treatment may suffer symptoms as noted in the DSM-5 evaluation criteria for major depressive disorder.

In embodiments, the present disclosure provides methods of treating major depressive disorder. The method comprising administering cariprazine or a pharmaceutically acceptable salt thereof in a therapeutically effective amount of 1.5 mg/day or 3 mg/day as an adjunct to antidepressant therapy to a patient in need of treatment for major depressive disorder, thereby treating said major depressive disorder.

In embodiments, the present disclosure provides method of treating major depressive disorder, the method comprising administering cariprazine or a pharmaceutically acceptable salt thereof in an amount of 1.5 mg/day or 3 mg/day to a patient in need of adjunctive therapy for major depressive disorder (MDD), wherein the administration of cariprazine achieves statistically significant change from baseline to Week 6 in the Montgomery Åsberg Depression Rating Scale (MADRS) total score.

In embodiments, the patient in need of adjunctive therapy for MDD meets Diagnostic and Statistical Manual of Mental Disorders, Fifth Edition (DSM-5) criteria for MDD based on Structured Clinical Interview for DSM-5 (SCID-5) and has a total score ≥22 on the Hamilton Depression Rating Scale-17 (HAMD-17) prior to treatment.

In embodiments, the patient has a major depressive episode of at least 8 weeks to less than 24 months.

In embodiments, the patient demonstrates inadequate response to 1 to 3 antidepressants of adequate dose and adequate duration as measured by Antidepressant Treatment Response Questionnaire (ATRQ).

In embodiments, the method of treating major depressive disorder comprises administering cariprazine or a pharmaceutically acceptable salt thereof in a therapeutically effective amount of 1.5 mg/day as an adjunct to antidepressant therapy to a patient in need of treatment for major depressive disorder for 6 weeks, thereby treating said major depressive disorder.

In embodiments, administration of cariprazine or a pharmaceutically acceptable salt in an amount of 1.5 mg/day in addition to antidepressant therapy results in a statistically significant remission of the depressive episode as defined by less than 10 total score on MADRS total score.

In embodiments, the statistically significant remission is achieved with less than 2% to no adverse events.

In embodiments, cariprazine or a pharmaceutically acceptable salt is administered as cariprazine hydrochloride.

In embodiments, the method comprises administering cariprazine or a pharmaceutically acceptable salt thereof in a therapeutically effective amount of 3.0 mg/day as an adjunct to antidepressant therapy to a patient in need of treatment for major depressive disorder for 6 weeks, thereby treating said major depressive disorder.

In embodiments, administration of cariprazine or a pharmaceutically acceptable salt in an amount of 3.0 mg/day in addition to antidepressant therapy results in a statistically significant remission of a major depressive episode as defined by less than 10 total score on MADRS total score.

In embodiments, the present disclosure provides a method of statistically significant treatment of major depressive disorder in patients who had inadequate responses to one or more antidepressants, comprising administering to each said patient cariprazine or a pharmaceutically acceptable salt thereof in an amount of 1.5 mg per day as an adjunctive therapy to one or more said antidepressants, and said treatment achieves statistically significant least square mean change in the MADRS total score in said patients from baseline to week 6 of said treatment as compared to placebo.

In embodiments, the method of statistically significant treatment of major depressive disorder in patients who had inadequate responses to one or more antidepressants, comprises administering to each said patient cariprazine or a pharmaceutically acceptable salt thereof in an amount of 1.5 mg per day as an adjunctive therapy to one or more said antidepressants, and said treatment achieves statistically significant least square mean change in the MADRS total score in said patients from baseline to week 6 of said treatment as compared to placebo, wherein said statistically significant least square mean change in the MADRS total score in said patients from baseline to week 6 of said treatment as compared to placebo is −2 points or exceeds said 2-points.

In embodiments, the present disclosure provides for improving or alleviating patient symptoms associated with major depressive disorder. Such patient symptom can be assessed or defined as an improvement in Patient Reported Outcomes or Clinician Reported Outcomes as further described herein. Improvements can affect patient symptoms of restlessness, fatigue, excessive anxiety and worry, increased muscle aches or soreness, impaired concentration, irritability, and difficulty sleeping, for example as outlined in the DSM-5.

The invention of the present disclosure will be more clearly understood by reference to the following examples, which are included for purposes of illustration only and are not intended to be limiting.

### Example 1

The study evaluated the efficacy, safety, and tolerability of cariprazine 1.5 mg/day and 3 mg/day compared with placebo as an adjunctive treatment to ongoing antidepressant therapy (ADT) in patients with major depressive disorder (MDD) who have had an inadequate response to antidepressants alone.

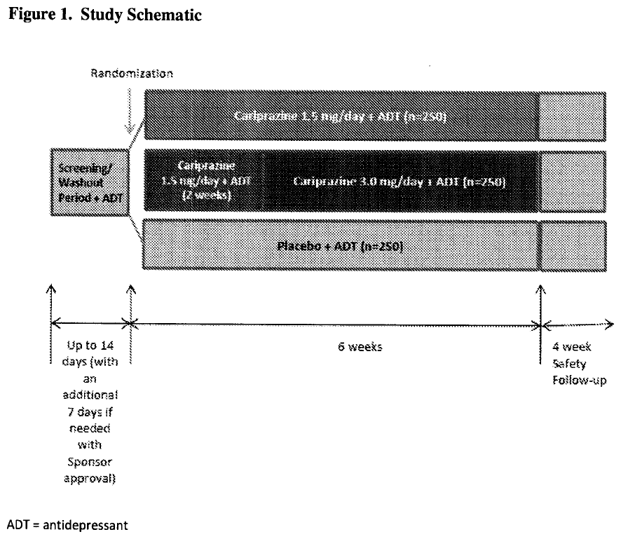

A global, multicenter, randomized, double-blind (DB), placebo-controlled, parallel-group, fixed-dose study compared cariprazine 1.5 mg/day and cariprazine 3 mg/day with placebo as an adjunctive treatment to ongoing ADT in outpatients with a diagnosis of MDD (via the Diagnostic and Statistical Manual of Mental Disorders, Fifth Edition [DSM-5]) having an inadequate response, as measured by the modified Antidepressant Treatment Response Questionnaire (ATRQ), to 1 to 3 antidepressants administered during the current episode at an adequate dose and adequate duration. Adequate dose was defined as a dose above the minimum labeled dose (per package insert). Adequate duration was defined as continuous ADT treatment for at least 6 weeks, with a minimum of 3 of 6 weeks above the minimal dose. The study schema is presented in FIG. 1.

The study population included patients meeting criteria for MDD with a current major depressive episode of at least 8 weeks to less than 24 months in duration and an inadequate response to ongoing antidepressant therapy on the current episode. Both male and female patients of 18 to 65 years of age qualified for the study. Patients met the DSM-5 criteria for MDD based on the SCID-5, with a current major depressive episode of at least 8 weeks to less than 24 months in duration at the time of patient screening. A diagnosis of MDD with psychotic features was considered acceptable.

In their current depressive episode, the patients must have had an inadequate response (<50% improvement) to 1 to 3 antidepressants of adequate dose and adequate duration as measured by the modified ATRQ. Adequate dose was defined as a dose above the minimum labeled dose (per package insert). Adequate duration was defined as continuous antidepressant therapy treatment for at least 6 weeks, with a minimum of 3 of 6 weeks above the minimum dose. Patients must have had a minimum score of 22 on the rater administered Hamilton Depression Rating Scale-17 items (HAMD-17) at both the screening (Visit 1) and baseline (Visit 2) visits. Patients also had a score of 2 or higher on Item 1 of the HAMD-17.

Patients with a diagnosis of any current psychiatric diagnosis other than MDD, with exception of specific phobias, were excluded. Patients with a history of meeting DSM-5 for substance-related disorders (i.e., use disorders except caffeine-and tobacco-related) and addictive disorders within the 6 months before first visit were also excluded.

The study consisted of up to 14 days screening (with up to an additional 7 days of needed) and washout of prohibited medications followed by 6 weeks of double-blind (DB) treatment followed by a 4-week safety follow-up. At the end of the screening period, patients meeting the entry criteria for the study were randomized (1:1:1) to 1 of 3 double-blind treatment groups. Study treatment groups were: cariprazine 1.5 mg/day+antidepressant therapy and cariprazine 3 mg/day+antidepressant therapy. The control group received matching placebo+antidepressant therapy.

Dosage/Dose Regimen: Investigational product (cariprazine or placebo) in the form of capsules packaged in blister packs will be provided. No investigational product was administered during the screening/washout period; patients continued the same antidepressant and dose they were on at screening. Patients were taking more than one antidepressant at screening, regardless of the indication, discontinued all other antidepressants prior to baseline (Visit 2). During the double-blind treatment period (6 weeks), patients were administered 1 capsule orally per day in addition to their ongoing ADT. Patients in the 1.5 mg/day+ADT arm were administered 1.5 mg+ADT starting at Visit 2 (Week 0). Patients in the 3 mg/day+ADT arm were administered 1.5 mg+ADT starting at Visit 2 (Week 0) for 2 weeks and then titrate to 3 mg/day+ADT starting at Visit 4 (Week 2). Patients were supplied with identically appearing capsules of either cariprazine 1.5 mg, cariprazine 3 mg, or placebo.

Safety Follow-up: After completion of the double-blind treatment period, patients continued as outpatients during the safety follow-up (FU) period and received treatment as usual at the discretion of the investigator or designee. No investigational product was administered after the treatment period.

### Clinical Study A

A randomized, double-blind, placebo-controlled, multicenter trial with 759 participants was conducted in United States, Bulgaria, Estonia, Germany, Hungary, Ukraine, and the United Kingdom. Following a screening period of up to 14 days (with up to an additional 7 days if needed), which included a washout of prohibited psychotropic medications except for ongoing ADT, patients with a partial response to their antidepressant monotherapy were randomized into three treatment groups (1:1:1). One group received cariprazine 1.5 mg/d+ADT, one received cariprazine 3.0 mg/d+ADT, and one group received placebo+ADT. All participants randomized to cariprazine began on 1.5 mg/d; cariprazine 1.5 mg/d patients remained at that dosage, while cariprazine 3.0 mg/d patients received 1.5 mg/d for 2 weeks and increased to 3.0 mg/d on day 15.

For six weeks, the medication was given once daily in addition to the ongoing ADT treatment, to which the patient had experienced inadequate clinical response. In Study 301, cariprazine met its primary endpoint and demonstrated statistically significant change from baseline to week 6 in the Montgomery-Åsberg Depression Rating Scale (MADRS) total score in a trial evaluating patients with major depressive disorder. Patients treated with cariprazine at 1.5 mg/day achieved improved MADRS total score at week six compared to placebo (p-value=0.0050). Patients treated with cariprazine at 3.0 mg/day demonstrated improvement in MADRS total score at week six over placebo but did not meet statistical significance (p-value=0.0727).

Study participant disposition, discontinuations, and demographics (safety population) and demographics (mITT population) are shown below in Tables 1, 2, 3, and 4 respectively.

TABLE 1
Disposition

| | | Cariprazine | Cariprazine | |
|---|---|---|---|---|
| | Placebo + | 1.5 mg/day + | 3 mg/day + | |
| Number of Participants | ADT | ADT | ADT | Total |
| Screened | | | | 1575 |
| Randomized | 254 | 252 | 253 | 759 |
| Treated (Safety population) | 253 (99.6%) | 252 (100%) | 252 (99.6%) | 757 (99.7%) |
| Modified Intent-to-treat | 249 (98.0) | 250 (99.2) | 252 (99.6) | 751 (98.9) |
| (mITT population) | | | | |
| Completed DB Period | 229 (90.2) | 231 (91.7) | 219 (86.6) | 679 (89.5) |
| Entered in Safety FU Period | 240 (94.9) | 239 (94.8) | 238 (94.4) | 717 (94.7) |
| Completed Safety FU Period | 237 (93.3) | 234 (92.9) | 230 (90.9) | 701 (92.4) |

TABLE 2
Premature Discontinuations (Randomized Population)

| | | Cariprazine | Cariprazine | |
|---|---|---|---|---|
| | | 1.5 | 3 mg/ | |
| | Placebo + | mg/day + | day + | |
| | ADT | ADT | ADT | Total |
| | (N = 254) | (N = 252) | (N = 253) | (N = 759) |
| Disposition | n (%) | n (%) | n (%) | n (%) |
| Completed | 229 (90.2) | 231 (91.7) | 219 (86.6) | 679 (89.5) |
| DB Period | | | | |
| Prematurely | 24 (9.5) | 21 (8.3) | 33 (13.1) | 78 (10.3) |
| Discontinued | | | | |
| Reasons for | | | | |
| Discontinuation | | | | |
| Adverse Event | 6 (2.4) | 3 (1-2) | 18 (7.1) | 27 (3.6) |
| Lack of Efficacy | 2 (0.8) | 2 (0.8) | 0 | 4 (0.5) |
| Withdrawal | 13 (5.1) | 11 (4.4) | 9 (3.6) | 33 (4.4) |
| by Subject | | | | |
| Lost to | 3 (1-2) | 3 (1-2) | 5 (2.0) | 11 (1.5) |
| Follow-Up | | | | |
| Pregnancy | 0 | 0 | 0 | 0 |
| Death | 0 | 0 | 0 | 0 |
| Protocol Deviation | 0 | 1 (0.4) | 0 | 1 (0.1) |
| Non-Compliance | 0 | 1 (0.4) | 1 (0.4) | 2 (0.3) |
| with Study Drug | | | | |
| Non-Compliance | 0 | 0 | 0 | 0 |
| with ADT | | | | |
| Study or Site | 0 | 0 | 0 | 0 |
| Terminated by | | | | |
| the Sponsor | | | | |
| Other | 0 | 0 | 0 | 0 |

TABLE 3
Demographics (safety population)

| | | Cariprazine 1.5 | Cariprazine3.0 |
|---|---|---|---|
| | Placebo + | mg/day + ADT | mg/day + ADT |
| | ADT | (N = 252) | (N = 252) |
| | (N = 253) | n (%) | n (%) |
| Age, mean | 46.4 (11.9) | 43.3 (13.6) | 44.8 (13.3) |
| (SD), years | | | |
| Female, n (%) | 184 (72.7) | 191 (75.8) | 180 (71.4) |

Race, n (%)

| | | | |
|---|---|---|---|
| White | 203 (80.2) | 205 (81.3) | 215 (85.3) |
| Asian | 5 (2.0) | 4 (1-6) | 7 (2.8) |
| Other | 2 (0.8) | 6 (2.4) | 0 (0.0) |
| BMI, mean | 30.5 (7.9) | 30.1 (7.6) | 29.0 (7.0) |
| (SD), kg/m² | | | |

Psychiatric history

| | | | |
|---|---|---|---|
| Duration of current | 8.3 (5.3) | 6.8 (4.3) | 7.9 (4.8) |
| episode of MDD, n | | | |
| (%), months | | | |
| Number of lifetime | 6.9 (19.8) | 6.2 (8.6) | 6.2 (7.2) |
| MDD episodes, | | | |
| mean (SD) | | | |
| Lifetime Duration | 14.8 (11.6) | 12.8 (10.7) | 14.0 (12.1) |
| of MDD, | | | |
| mean (SD), years | | | |

Baseline Efficacy Variables

| | | | |
|---|---|---|---|
| MADRS total score, | 31.9 (5.7) | 32.8 (5.0) | 32.7 (4.9) |
| mean (SD) | | | |

TABLE 4
Demographics (mITT population)

| | | | Cari- | Cari- | |
|---|---|---|---|---|---|
| | | | prazine | prazine | |
| | | | 1.5 mg/ | 3.0 mg/ | |
| | | Placebo + | day + | day + | |
| | | ADT | ADT | ADT | Total |
| | | (N = 249) | (N = 250) | (N = 252) | (N = 751) |
| | | n (%) | n (%) | n (%) | n (%) |
| Age | Mean | 46.5 | 43.2 | 44.8 | 44.8 |
| (years): | (SD) | (11.9) | (13.6) | (12.5) | (13.0) |
| | Min, Max | 20, 65 | 18, 65 | 18, 65 | 18, 65 |
| Sex, | Male | 68 (27.3) | 60 (24.0) | 72 (28.6) | 200 (26.6) |
| n (%): | Female | 181 (72.7) | 190 (76.0) | 180 (71.4) | 551 (73.4) |
| Race, | White | 200 (80.3) | 203 (81.2) | 215 (85.3) | 618 (82.3) |
| n (%): | | | | | |
| Ethnicity, | Hispanic | 25 (10.0) | 24 (9.6) | 19 (7.5) | 68 (9.1) |
| n (%): | Non- | 224 (90.0) | 226 (90.4) | 233 (92.5) | 683 (90.9) |
| | Hispanic | | | | |
| Region, | US | 148 (59.4) | 151 (60.4) | 153 (60.7) | 452 (60.2) |
| n (%): | Non-US | 101 (40.6) | 99 (39.6) | 99 (39.3) | 299 (39.8) |

The primary and secondary efficacy parameters were change from baseline to week 6 in Montgomery-Åsberg Depression Rating Scale (MADRS) total score and Clinical Global Impressions-Severity (CGI-S) score, respectively (screening, baseline, and weeks 1, 2, 4, and 6). Additional efficacy parameters (screening, baseline, and ≥1 double-blind visit) included: MADRS response (≥50% MADRS total score reduction) and remission (MADRS total score ≤10); change from baseline in HAMD-17 total score and Hamilton Anxiety Rating Scale (HAM-A) total score (32); and Clinical Global Impressions-Improvement (CGI-I) score and response (CGI-I score ≤2).

Of 1575 patients screened for eligibility, 759 were randomized to double-blind treatment, 757 were included in the safety population, and 751 were included in the mITT population.

Mean (SD) MADRS scores at week 6 were 19.5 (10.3) for placebo, 17.4 (9.1) for cariprazine 1.5 mg/d, and 18.6 (8.9) for cariprazine 3 mg/d. Adjunctive cariprazine 1.5 mg/d resulted in significantly greater mean reductions from baseline to week 6 in MADRS total score versus placebo (−14.1 versus −11.5; p=0.0025; adjusted p=0.0050). MADRS score reductions were significant versus placebo for cariprazine 1.5 mg/d at week 2 (nominal p=0.0453) and week 4 (nominal p<0.0001). Compared with placebo, cariprazine 3.0 mg/d resulted in numerically greater reductions in MADRS total score from baseline to week 6 (−11.5 versus −13.1); however, these differences did not reach statistical significance (nominal p=0.0691; adjusted p=0.0727). Results of the sensitivity analysis were consistent with the primary results.

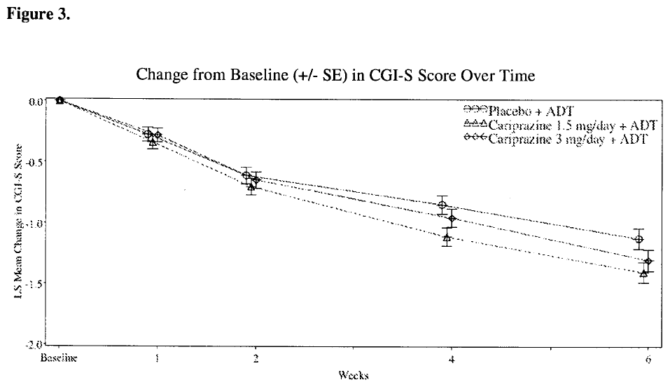

Change from baseline (+/−SE) in CGI-S Score over time is shown in FIG. 3. The difference in change from baseline to week 6 in CGI-S score (secondary efficacy) was significant for cariprazine 1.5 mg/d versus placebo (nominal p=0.0091); significance was not retained after adjusting for multiplicity (adjusted p=0.0727). Greater reductions in CGI-S score for cariprazine 1.5 mg/d versus placebo were observed starting at week 4 (nominal p=0.0033) and maintained through week 6. Numerically greater CGI-S reductions were also seen for cariprazine 3.0 mg/d versus placebo from week 4 through 6, though these differences did not achieve statistical significance.

Efficacy parameters are summarized in Table 5. At week 6, rates of MADRS response were significantly greater for cariprazine 1.5 mg/d versus placebo (44.0% versus 34.9%; nominal p=0.0446). There were no significant differences in MADRS remission rates versus placebo (23.3%) for either cariprazine dose (1.5 mg/d: 25.2% [nominal p=0.3691]; 3 mg/d: 16.7% [p=0.1155]). At week 6, HAMD-17 total score reduction was significantly greater for cariprazine 1.5 mg/d versus placebo (−12.7 versus −10.6; nominal p=0.0014), but not for cariprazine 3.0 mg/day (−11.9; p=0.0597). At week 6, the difference in HAM-A scores was significant in favor of cariprazine 1.5 mg/d versus placebo (−9.1 versus −7.8; nominal p=0.0370). Compared with placebo, greater improvement as measured by CGI-I scores at week 6 was observed for both cariprazine 1.5 mg/d (nominal p=0.0026) and cariprazine 3.0 mg/d (nominal p=0.0076); numerically higher CGI-I responder rates were observed for cariprazine 1.5 mg/d (51.2%) and 3.0 mg/d (50.4%) versus placebo (43.0%), but the differences did not achieve nominal significance.

TABLE 5
Efficacy Parameters, Response, and Remission (mITT Population)

| | | | | Week 6 | | | | | |
|---|---|---|---|---|---|---|---|---|---|
| | | | | Change | | Difference Versus Placebo | | | |
| | Baseline | | | LS | | | | | Adjusted |
| | N | Mean | SD | Mean | SE | LSMD | 95% CI^{a} | p value^{a} | p value^{b} |
| Primary Efficacy Parameter: MADRS | | | | | | | | | |
| MMRM | | | | | | | | | |
| Placebo + ADT | 249 | 31.9 | 5.7 | −11.5 | 0.7 | — | — | — | — |
| CAR 1.5 mg/d + ADT | 250 | 32.8 | 5.0 | −14.1 | 0.7 | −2.5 | −4.2, −0.9 | 0.0025 | 0.0050 |
| CAR 3.0 mg/d + ADT | 252 | 32.7 | 4.9 | −13.1 | 0.7 | −1.5 | −3.2, 0.1 | 0.0691 | 0.0727 |
| ANCOVA and LOCF^{c} | | | | | | | | | |
| Placebo + ADT | 249 | 31.9 | 5.7 | −12.0 | 0.9 | — | — | — | — |
| CAR 1.5 mg/d + ADT | 250 | 32.8 | 5.0 | −14.4 | 0.9 | −2.4 | −4.0, −0.8 | 0.0032 | — |
| CAR 3.0 mg/d + ADT | 252 | 32.7 | 4.9 | −13.3 | 0.9 | −1.3 | −2.9, 0.3 | 0.1229 | — |
| Secondary Efficacy Parameter: CGI-S | | | | | | | | | |
| MMRM | | | | | | | | | |
| Placebo + ADT | 249 | 4.6 | 0.6 | −1.1 | 0.1 | — | — | — | — |
| CAR 1.5 mg/d + ADT | 250 | 4.6 | 0.6 | −1.4 | 0.1 | −0.3 | −0.5, −0.1 | 0.0091 | 0.0727 |
| CAR 3.0 mg/d + ADT | 252 | 4.6 | 0.6 | −1.3 | 0.1 | −0.2 | −0.4, 0.0 | 0.0944 | 0.0944 |
| Additional Efficacy Parameters | | | | | | | | | |
| HAMD-17 | | | | | | | | | |
| ANCOVA and LOCF^{c} | | | | | | | | | |
| Placebo + ADT | 249 | 25.6 | 2.7 | −10.6 | 0.7 | — | — | — | — |
| CAR 1.5 mg/d + ADT | 250 | 25.9 | 2.7 | −12.7 | 0.7 | −2.1 | −3.3, −0.8 | 0.0014 | — |
| CAR 3.0 mg/d + ADT | 252 | 26.2 | 2.9 | −11.9 | 0.7 | −1.2 | −2.5, 0.1 | 0.0597 | — |
| HAM-A | | | | | | | | | |
| MMRM | | | | | | | | | |
| Placebo + ADT | 249 | 20.6 | 7.4 | −7.8 | 0.6 | — | — | — | — |
| CAR 1.5 mg/d + ADT | 250 | 21.7 | 6.9 | −9.1 | 0.6 | −1.3 | −2.5, −0.1 | 0.0370 | — |
| CAR 3.0 mg/d + ADT | 252 | 21.9 | 7.8 | −8.6 | 0.6 | −0.8 | −2.0, 0.5 | 0.2219 | — |
| CGI-I | | | | | | | | | |
| MMRM | | | | | | | | | |
| Placebo + ADT | 249 | — | — | 2.8 | 0.1 | — | — | — | — |
| CAR 1.5 mg/d + ADT | 250 | — | — | 2.6 | 0.1 | −0.3 | −0.5, −0.1 | 0.0026 | — |
| CAR 3.0 mg/d + ADT | 252 | — | — | 2.6 | 0.1 | −0.3 | −0.5, −0.1 | 0.0076 | — |

| | | | Odds Ratio Versus Placebo (LOCF) | | |
|---|---|---|---|---|---|
| | N | % | OR | 95% CI | p value |
| MADRS response (≥50% score | | | | | |
| reduction from baseline) | | | | | |
| Placebo + ADT (N = 249) | 87 | 34.9 | — | — | — |
| CAR 1.5 mg/d + ADT (N = 250) | 110 | 44.0 | 1.5 | 1.0, 2.1 | 0.0446 |
| CAR 3.0 mg/d + ADT (N = 252) | 99 | 39.3 | 1.2 | 0.8, 1.7 | 0.3409 |
| MADRS remission (total score ≤10) | | | | | |
| Placebo + ADT (N = 249) | 58 | 23.3 | — | — | — |
| CAR 1.5 mg/d + ADT (N = 250) | 63 | 25.2 | 1.2 | 0.8, 1.8 | 0.3691 |
| CAR 3.0 mg/d + ADT (N = 252) | 42 | 16.7 | 0.7 | 0.4, 1.1 | 0.1155 |
| CGI-I response (score ≤2) | | | | | |
| Placebo + ADT (N = 249) | 107 | 43.0 | — | — | — |
| CAR 1.5 mg/d + ADT (N = 250) | 128 | 51.2 | 1.4 | 1.0, 2.0 | 0.0627 |
| CAR 3.0 mg/d + ADT (N = 252) | 127 | 50.4 | 1.4 | 1.0, 1.9 | 0.0935 |
| HAM-A response (≥50% score | | | | | |
| reduction from baseline) | | | | | |
| Placebo + ADT (N = 247) | 87 | 35.2 | — | — | — |
| CAR 1.5 mg/d + ADT (N = 247) | 101 | 40.9 | 1.3 | 0.88, 1.83 | 0.2052 |
| CAR 3.0 mg/d + ADT (N = 251) | 103 | 41.0 | 1.3 | 0.88, 1.83 | 0.2029 |
| HAM-A remission (total score ≤7) | | | | | |
| Placebo + ADT (N = 247) | 67 | 27.1 | — | — | — |
| CAR 1.5 mg/d + ADT (N = 247) | 71 | 28.7 | 1.2 | 0.79, 1.78 | 0.4122 |
| CAR 3.0 mg/d + ADT (N = 251) | 70 | 27.9 | 1.1 | 0.76, 1.71 | 0.5306 |

^{a}p value and 95% confidence interval for the difference using contrast t-test.
^{b}Adjusted p values: The truncated Hochberg procedure with truncation parameter of 0.9 was used for the primary endpoint and the regular Hochberg for the key secondary endpoint to control the overall type I error rate at a 0.05 level (2 sided) for multiple comparisons of 2 active doses with placebo for the primary and key secondary endpoint.
^{c}The p value for a between-treatment comparison at each visit is based on a logistic regression model which included treatment, country, ADT failure category, and corresponding baseline value as explanatory variables.
LOCF was used for imputation.
ADT = antidepressant therapy; ANCOVA = analysis of covariance; CAR = cariprazine; CGI-I = Clinical Global Impressions-Improvement; CGI-S = Clinical Global Impressions-Severity; CI = confidence interval; HAM-A = Hamilton Rating Scale for Anxiety; HAMD-17 = 17-item Hamilton Depression Rating Scale; LOCF = last-observation carried forward; LS = least squares; LSMD = least squares mean difference; MADRS = Montgomery-Åsberg Depression Rating Scale; mITT = modified intent-to-treat; MMRM = Mixed Model Repeated Measures; OR = odds ratio; SD = standard deviation; SE = standard error.

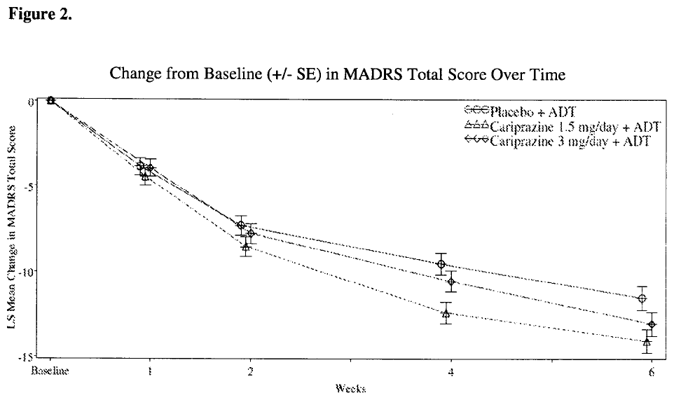

Results of study participant response in MADRS total score and MADRS over time are shown in Tables 6 and 7, respectively. Analysis was conducted using mixed model for repeated measures (MMRM) method. Statistically significant least square mean change in the MADRS total score in patients from baseline to week 6 of treatment as compared to placebo was reported as least square mean difference (LSMD) versus placebo. A 2-point or more change in the LSMD in MADRS total score for treatment versus placebo has been commonly used to indicate a clinically relevant treatment effect. Table 8 summarizes the number of study participants who were MADRS responders or achieved MADRS remission (MADRS total score ≤10). Results of change from baseline (+/− standard error (SE)) MADRS total score over time are shown in FIG. 2.

TABLE 6
Change from Baseline, MADRS Total Score at Week 6 (MMRM)

| | Placebo + | CAR 1.5 | CAR 3 |
|---|---|---|---|
| | ADT | mg/day + ADT | mg/day + ADT |
| | (N = 249) | (N = 250) | (N = 252) |
| Baseline, Mean (SD) | 31.9 (5.7) | 32.8 (5.0) | 32.7 (4.9) |
| WEEK 6 | | | |
| n | 231 | 231 | 223 |
| Least Square | −11.5 (0.7) | −14.1 (0.7) | −13.1 (0.7) |
| Mean (SE) | | | |
| LSMD vs | | −2.5 | −1.5 |
| Placebo (95% CI) | | (−4.2, −0.9) | (−3.2, 0.1) |
| Nominal p-value | | 0.0025 | 0.0691 |
| Adjusted p-value | | 0.0050 | 0.0727 |

TABLE 7
Change from Baseline, MADRS Total Score over time (MMRM)

| | | Cariprazine | Cariprazine |
|---|---|---|---|
| | Placebo + ADT | 1.5 mg/day + | 3 mg/day + |
| Change from | (N = 249) | ADT (N = 250) | ADT (N = 252) |
| Baseline | LS Mean (SE) | LS Mean (SE) | LS Mean (SE) |
| Week 1 | −3.8 (0.5) | −4.4 (0.5) | −3.9 (0.5) |
| Week 2 | −7.3 (0.6) | −8.6 (0.6) | −7.8 (0.6) |
| Week 4 | −9.5 (0.6) | −12.4 (0.6) | −10.6 (0.6) |
| Week 6 | −11.5 (0.7) | −14.1 (0.7) | CGI-I (0.7) |

| | LSMD (95% CI): Nominal P-value | |
|---|---|---|
| Comparisons | Cariprazine 1.5 | Cariprazine 3 mg/ |
| between | mg/day + ADT | day + ADT vs. |
| Treatments | vs. Placebo + ADT | Placebo + ADT |
| Week 1 | −0.6 (−1.5; 0.5) | −0.1 (−1.0; 0.8) |
| | 0.2123 | 0.8301 |
| Week 2 | −1.2 (−2.5; 0) | −0.5 (−1.7; 0.8) |
| | 0.0453 | 0.4512 |
| Week 4 | −2.9 (−4.3; −1.5) | −1.0 (−2.4; 0.4) |
| | <0.0001 | 0.1530 |
| Week 6 | −2.5 (−4.2; −0.9) | −1.5 (−3.2; 0.1) |
| | 0.0025 | 0.0691 |

TABLE 8
MADRS Responders and Remissions

| | | |
|---|---|---|
| | CAR | |
| | 1.5 mg/ | CAR 3 |
| Placebo + | day + | mg/day + |
| ADT | ADT | ADT |
| (N = 249) | (N = 250) | (N = 251) |

MADRS RESPONDERS (Total score ≥50%)

| | | | |
|---|---|---|---|
| Week 2, n (%) | 36 (14.5) | 43 (17.2) | 39 (15.5) |
| Odds ratio vs | | 1.2 (0.7, 2.0) | 1.1 (0.7, 1.8) |
| placebo (95% CI) | | | |
| p-value | | 0.4344 | 0.7945 |
| Week 4, n (%) | 64 (25.7) | 88 (35.2) | 60 (23.8) |
| Odds ratio vs | | 1.6 (1.1, 2.3) | 0.9 (0.6, 1.3) |
| placebo (95% CI) | | | |
| p-value | | 0.0265 | 0.5773 |
| Week 6, n (%) | 87 (34.9) | 110 (44.0) | 99 (39.3) |
| Odds ratio vs | | 1.5 (1.0, 2.1) | 1.2 (0.8, 1.7) |
| placebo (95% CI) | | | |
| p-value | | 0.0446 | 0.3409 |

MADRS REMISSIONS (Total score ≤10)

| | | | |
|---|---|---|---|
| Week 2, n (%) | 18 (7.2) | 13 (5.2) | 9 (3.6) |
| Odds ratio vs | | 0.9 (0.4, 1.8) | 0.5 (0.25, 1.23) |
| placebo (95% CI) | | | |
| p-value | | 0.6816 | 0.1463 |
| Week 4, n (%) | 35 (14.1) | 37 (14.8) | 22 (8.7) |
| Odds ratio vs | | 1.2 (0.7, 1.9) | 0.6 (0.4, 1.1) |
| placebo (95% CI) | | | |
| p-value | | 0.5662 | 0.0931 |
| Week 6, n (%) | 58 (23.3) | 63 (25.2) | 42 (16.7) |
| Odds ratio vs | | 1.2 (0.8, 1.8) | 0.7 (0.4, 1.1) |
| placebo (95% CI) | | | |
| p-value | | 0.3691 | 0.1155 |

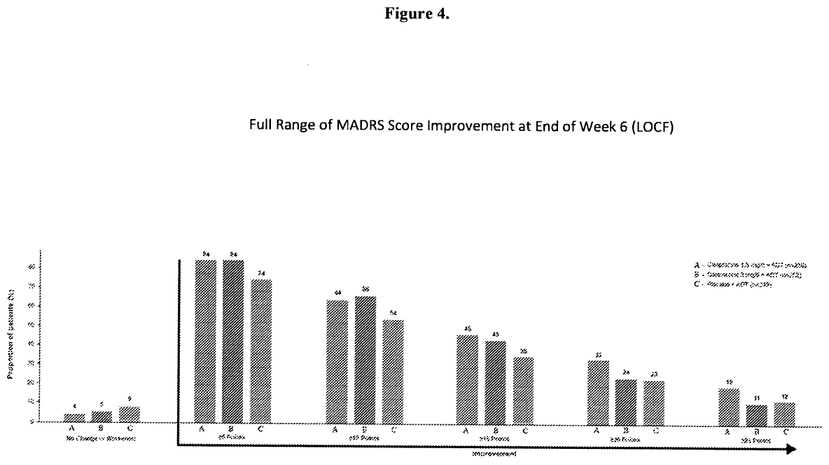

In post hoc analysis, a greater proportion of patients receiving cariprazine+ADT versus placebo+ADT improved ≥5 points (1.5 mg/d and 3.0 mg/d=84%; placebo=74%), ≥10 points (1.5 mg/d=64%; 3.0 mg/d=66%; placebo=54%), and ≥15 points (1.5 mg/d=46%; 3.0 mg/d=43%; placebo=35%). More cariprazine 1.5 mg/d patients versus placebo patients improved ≥20 points (1.5 mg/d=33%; 3.0 mg/d=24%; placebo=23%) and ≥25 points (1.5 mg/d=19%; 3.0 mg/d=11%; placebo=12%). FIG. 4 shows the full range of MADRS score improvement at the end of week 6 (LOCF) by proportion of patients (%).

The LSMD (95% CI) for MADRS individual term score change from baseline to week 6 was statistically significant in favor of CAR vs. placebo for “apparent sadness” (−0.2 [−0.44, −0.03], p=0.0247), “reported sadness” (−0.4 [−0.58, −0.16], p=0.0006), “reduced appetite” (−0.3 [−0.50, −0.10], p=0.0036), “lassitude” (−0.3 [0.56, −0.12], p=0.0025), “inability to feel” (−0.3 [−0.51, −0.06, p=0.0126), “pessimistic thoughts” (−0.3 [−0.45, −0.07], p=0.0088), and “suicidal thoughts” (−0.1 [−0.20, −0.02], p=0.0127).

Results of study participant response in Clinical Global Impressions-Severity (CGI-S) score at Week 6 and CGI-S score over time are shown in Tables 9 and 10, respectively. The number of Responders (Score ≤2) on the Clinical Global Impressions-Improvement (CGI-I) scale are shown on Table 11 (A and B). Results of CGI-S score over time are shown in FIG. 3.

TABLE 9
Change from Baseline, CGI-S Score at Week 6 (MMRM)

| | Placebo + | CAR 1.5 mg/ | CAR 3 mg/ |
|---|---|---|---|
| | ADT | day + ADT | day + ADT |
| | (N = 249) | (N = 250) | (N = 252) |
| Baseline, mean (SD) | 4.6 (0.6) | 4.6 (0.6) | 4.6 (0.6) |
| WEEK 6 | | | |
| n | 231 | 231 | 223 |
| Least Square | −1.1 (0.1) | −1.4 (0.1) | −1.3 (0.1) |
| Mean (SE) | | | |
| LSMD vs | | −0.3 (−0.5, −0.1) | −0.2 (−0.4, 0) |
| Placebo (95% CI) | | | |
| Nominal p-value | | 0.0091 | 0.0944 |
| Adjusted p-value | | 0.0727 | 0.0944 |

TABLE 10
Change from Baseline, CGI-S Score overtime (MMRM)

| | | Cariprazine | Cariprazine |
|---|---|---|---|
| | | 1.5 mg/day + | 3 mg/day + |
| | Placebo + ADT | ADT | ADT |
| Change from | (N = 249) | (N = 250) | (N = 252) |
| Baseline | LS Mean (SE) | LS Mean (SE) | LS Mean (SE) |
| Week 1 | −0.3 (0.1) | −0.3 (0.1) | −0.3 (0.1) |
| Week 2 | −0.6 (0.1) | −0.7 (0.1) | −0.6 (0.1) |
| Week 4 | −0.9 (0.1) | −1.1 (0.1) | −1.0 (0.1) |
| Week 6 | −1.1 (0.1) | −1.4 (0.1) | −1.3 (0.1) |

| | LSMD (95% CI) | |
|---|---|---|
| Comparisons | Nominal P−value | |
| between | Cariprazine 1.5 mg/ | Cariprazine 3 mg/ |
| Treatment | day + ADT vs. | day + ADT vs. |
| Groups | Placebo + ADT | Placebo + ADT |
| Week 1 | −0.1 (−0.2, 0) | 0 (−0.1, 0.1) |
| | 0.2444 | 0.8975 |
| Week 2 | −0.1 (−0.2, 0.1) | 0 (−0.2, 0.1) |
| | 0.2008 | 0.6114 |
| Week 4 | −0.3 (−0.4, −0.1) | −0.1 (−0.3, 0.1) |
| | 0.0033 | 0.2261 |
| Week 6 | −0.3 (−0.5, −0.1) | −0.2 (−0.4, 0) |
| | 0.0091 | 0.0944 |

TABLE 11
(A) Clinical Global Impressions-Improvement (CGI-I) Responders

| | | CAR | CAR |
|---|---|---|---|
| | Placebo + | 1.5 mg/ | 3 mg/ |
| | ADT | day + ADT | day + ADT |
| | (N = 249) | (N = 250) | (N = 252) |
| CGI-1 RESPONDERS | | | |
| (Score ≤2) | | | |
| Week 1, n (%) | 15 (6.1) | 29 (11.6) | 20 (7.9) |
| Odds ratio vs | | 2.0 (1.0, 3.8) | 1.3 (0.6, 2.6) |
| placebo (95% CI) | | | |
| p-value | | 0.0425 | 0.4942 |
| Week 2, n (%) | 52 (20.9) | 67 (26.8) | 62 (24.6) |
| Odds ratio vs | | 1.4 (0.9, 2.1) | 1.2 (0.8, 1.9) |
| placebo (95% CI) | | | |
| p-value | | 0.1423 | 0.3730 |
| Week 4, n (%) | 87 (34.9) | 115 (46.0) | 102 (40.5) |
| Odds ratio vs | | 1.6 (1.1, 2.3) | 1.3 (0.9, 1.8) |
| placebo (95% CI) | | | |
| p-value | | 0.0120 | 0.2063 |
| Week 6, n (%) | 107 (43.0) | 128 (51.2) | 127 (50.4) |
| Odds ratio vs | | 1.4 (1.0, 2.0) | 1.4 (1.0, 1.9) |
| placebo (95% CI) | | | |
| p-value | | 0.0627 | 0.0935 |

TABLE 11
(B) CGI-I Efficacy Parameter

| | | | Cariprazine | Cariprazine 3 |
|---|---|---|---|---|
| | | Placebo + | 1.5 mg/d + | mg/d + |
| | | ADT | ADT | ADT |
| | | (n = 249) | (n = 250) | (n = 252) |
| MMRM | LS Mean (SE) change | 2.8 | 2.6 | 2.6 |
| | from baseline | (0.1) | (0.1) | (0.1) |
| | to week 6 | | | |
| | LSMD (95% CI) | | −0.3 | −0.3 |
| | | | (−0.5, −0.1) | (−0.5, −0.1) |
| | P value | | 0.0026 | 0.0076 |

Results of study participant response in the Hamilton Anxiety Rating Scale (HAM-A) and Hamilton Depression Rating Scale-17 (HAMD-17) are shown in Tables 12 and 13, respectively.

TABLE 12
Hamilton Anxiety Rating Scale (HAM-A)

| | | | Cariprazine | Cariprazine |
|---|---|---|---|---|
| | | Placebo + | 1.5 mg/d + | 3 mg/d + |
| | | ADT | ADT | ADT |
| | | (n = 249) | (n = 250) | (n = 252) |
| MMRM | LS Mean (SE) change | −7.8 | −9.1 | −8.6 |
| | from baseline | (0.6) | (0.6) | (0.6) |
| | to week 6 | | | |
| | LSMD (95% CI) | | −1.3 | −0.8 |
| | | | (−2.5, −0.1) | (−2.0, 0.5) |
| | P Value | | 0.0370 | 0.2219 |

TABLE 13
Hamilton Depression Rating Scale −17 (HAMD-17)

| | | | Cariprazine | Cariprazine |
|---|---|---|---|---|
| | | Placebo + | 1.5 mg/d + | 3 mg/d + |
| | | ADT | ADT | ADT |
| | | (n = 249) | (n = 250) | (n = 252) |
| ANCOVA | LS Mean (SE) change | −10.6 | −12.7 | −11.9 |
| and | from baseline | (0.7) | (0.7) | (0.7) |
| LOCF^{a} | to week 6 | | | |
| | LSMD (95% CI) | | −2.1 | −1.2 |
| | | | (−3.3, −0.8) | (−2.5, 0.1) |
| | P value | | 0.0014 | 0.0597 |

^{a}Based on ANCOVA (analysis of covariance) model using observed cases, with treatment group, pooled country, and ADT failure category as factors and baseline HAMD-17 total score as a covariate for between-treatment-group comparisons at week 6.

The number of participants (n) and percentage of participants ((%)) experiencing Adverse Events are shown in Table 14 below. Table 15 summarizes the most frequent TEAEs occurring in ≥5% in any treatment group, and Table 16 summarizes changes in weight. An AE was considered a treatment-emergent adverse event (TEAE) if the AE began or worsened (increased in severity or became serious) on or after the first dose of double-blinded investigational product.

TABLE 14
Adverse Events during Double-Blind Treatment Period

| | | Cariprazine | Cariprazine |
|---|---|---|---|
| | Placebo + | 1.5 mg/ | 3.0 mg/day |
| | ADT | day + ADT | + ADT |
| | (N = 253) | (N = 252) | (N = 252) |
| | n (%) | n (%) | n (%) |
| Treatment-emergent | 93 (36.8) | 125 (49.6) | 124 (49.2) |
| adverse events (TEAEs) | | | |
| Treatment-emergent | 2 (0.8) | 1 (0.4) | 1 (0.4) |
| Serious AE (TESAE) | | | |
| TEAE leading | 6 (2.4) | 3 (1-2) | 18 (7.1) |
| to discontinuation | | | |
| from study | | | |
| Any Extrapyramidal | 8 (3.2) | 25 (9.9) | 38 (15.1) |
| Symptoms (EPS) | | | |
| TEAEs | | | |
| Deaths | 0 | 0 | 0 |

TABLE 15
Most frequent TEAEs (≥5% in any treatment group)

| | Placebo + | Cariprazine | Cariprazine |
|---|---|---|---|
| | ADT | 1.5 mg/day + | 3.0 mg/day + |
| | N = 253 | ADT N = 252 | ADT N = 252 |
| | n (%) | n (%) | n (%) |
| Akathisia | 2 (0.8) | 13 (5.2) | 20 (7.9) |
| Nausea | 6 (2.4) | 20 (7.9) | 16 (6.3) |
| Insomnia | 10 (4.0) | 18 (7.1) | 16 (6.3) |
| Headache | 15 (5.9) | 22 (8.7) | 11 (4.4) |
| Somnolence | 7 (2.8) | 13 (5.2) | 11 (4.4) |

TABLE 16
Weight changes

| | | Cariprazine | Cariprazine |
|---|---|---|---|
| | Placebo | 1.5 mg + | 3 mg + |
| | (n = 253) | ADT (n = 252) | ADT (n = 252) |
| Change from | 0.11 (1.9) | 0.68 (2.5) | 0.78 (2.8) |
| baseline, mean | | | |
| (SD), kg | | | |
| Increase ≥7% from | 2 (0.8) | 10 (4.0) | 3 (1-2) |
| baseline, n (%), kg | | | |

Safety results of cariprazine were consistent with its established safety profile across indications with no new safety signals identified. The most common adverse events occurring at ≥5% in the cariprazine groups during the six-week study period were akathisia, nausea, insomnia, headache and somnolence. Changes in weight at the end of treatment were relatively small (<1 kg) in all treatment groups.

### Response Measures

### The Structured Clinical Interview for DSM-5 (SCID-5)

The SCID-5 is a semi-structured interview guide for making the major DSM-5 diagnoses (formerly diagnosed on Axis I). This clinician-rated diagnostic assessment is administered by an investigator, subinvestigator, or rater who has extensive professional training and experience in the diagnosis of mental illness. The SCID-5 was considered a source document for the study.

### The Modified Antidepressant Treatment Response Questionnaire

The ATRQ (Fava 2003) is a clinician-administered questionnaire that was used to determine whether the patient meets inclusion criteria for prior ADT treatment and response requirements. A modified ATRQ, completed by a clinician at the study site who was certified in administration of the ATRQ, was used to assess prior antidepressant exposure and response within the current depressive episode. The clinician identified the antidepressants the patient had previously taken within the current episode; indicating the dose range and duration. The clinician then selected the level of response the patient had to the antidepressant which resulted in the greatest response.

### Montgomery-Åsberg Depression Rating Scale

The MADRS (Montgomery and Åsberg, 1979) is a 10-item, clinician-rated scale that evaluates the patient's depressive symptomatology during the past week. Patients are rated on items assessing feelings of sadness, lassitude, pessimism, inner tension, suicidality, reduced sleep or appetite, difficulty in concentration, and lack of interest. Each item is scored on a 7-point scale with a score of 0 reflecting no symptoms and a score of 6 reflecting symptoms of maximum severity.

### The Hamilton Anxiety Rating Scale

The Hamilton Anxiety Rating Scale (HAM-A) (Hamilton 1959) is a clinician-rated scale which consists of 14 items, each rated on a 5-point scale ranging from 0 (not present) to 4 (very severe). The highest possible score is 56, which represents the most severe form of anxiety; the lowest possible score is 0, which represents an absence of anxiety. The instrument was administered by an experienced rater meeting the training requirements and qualifications set by the Sponsor.

The HAM-A is a 14-item, clinician-reported measure used to quantify and categorize the subject's anxiety over the past week. Items are rated on a 5-point Likert rating scale of “absent” (0; if symptoms are absent, insignificant, or clearly due to causes other than anxiety); “mild” (1; if symptom is infrequent, with no impairment and no more than mild distress); “moderate” (2; if symptom is more frequent, with moderate distress or limited interference with usual activities); “severe” (3; if symptom is severe and persistent or results in severe distress or marked impairment in functioning); “very severe” (4; if symptom is incapacitating). The HAM-A total score ranges from 0 to 56, with higher scores indicating greater anxiety severity. The HAM-A total score can further be divided into 7-item psychic anxiety cluster and somatic anxiety cluster scores ranging from 0 to 28 each; higher scores indicate greater anxiety severity. The HAM-A total score is used to stratify subjects by anxiety severity: 0 to 7 indicates no/minimal disease, 8 to 14 indicates mild disease, 15 to 23 indicates moderate disease, and ≥24 indicates severe disease.

### Clinical Global Impressions-Severity

The CGI-S (Guy 1976) is a clinician-rated scale that measures the overall severity of a patient's illness in comparison with the severity in other patients the physician has observed. The patient is rated on a scale from 1 to 7 with 1 indicating a “normal state” and 7 indicating “among the most extremely ill patients”. The CGI-S was administered by an investigator, subinvestigator or rater with extensive professional training and experience in assessing mental illness.

### Clinical Global Impressions-Improvement

The Clinical Global Impressions Improvement (CGI-I) scale (Guy 1976) is a clinician-rated scale that in this study was used to rate total improvement or worsening of mental illness from Visit 2, regardless of whether the investigator considered it to be a result of drug treatment or not. The patient was rated on a scale from 1 to 7, with 1 indicating that the patient was very much improved and 7 indicating that the patient was very much worse. The CGI-I was administered by an investigator, subinvestigator or rater with extensive professional training and experience in assessing mental illness.

### The Hamilton Depression Rating Scale-17 Items

The HAMD-17 (Hamilton 1960; Hamilton 1967; Miller et al, 1985) is a clinician-rated, 17-item scale used to rate the patient's depressive state based on feelings of depression, guilt, suicidality, anxiety, agitation, level of insight, patterns of insomnia, loss of interest in work and other activities, weight loss, hypochondriasis, and degree of psychomotor retardation. It also can be used to identify genital and somatic symptoms. This instrument was administered by an experienced rater meeting the training requirements and qualifications set by the Sponsor.

The HAMD-17 is a 17-item, clinician-reported measure used to quantify and categorize the subject's depression over the past week. Items are rated on varying numeric rating scales, ranging from 0 to 2 or 0 to 4. Each numeric rating is associated with a unique verbal anchor. The HAMD-17 total score ranges from 0 to 52 with higher scores indicating greater depression severity. The HAMD-17 total score is used to stratify subjects by depression severity: 0 to 7 indicates no/minimal disease, 8 to 13 indicates mild disease, 14 to 18 indicates moderate disease, 19 to 22 indicates severe disease, and ≥23 indicates very severe disease.

### References

Hamilton M., The assessment of anxiety states by rating. Br J Med Psychol. 1959; 32(1):50-55.

Hamilton M., A rating scale for depression. J Neurol Neurosurg Psychiatry 1960; 23:56-62.

Hamilton M., Development of a rating scale for primary depressive illness. Br J Soc Clin Psychol. 1967; 6(4):278-296.

Guy W., ECDEU assessment manual for psychopharmacology—revised. DHEW publication no. ADM 76-338. Rockville, MD: US Department of Health, Education, and Welfare; Public Health Service; Alcohol, Drug Abuse, and Mental Health Administration; National Institute of Mental Health; Psychopharmacology Research Branch; Division of Extramural Research Programs; 1976; 217-222 (CGI); 534-537 (AIMS).

Montgomery S A, Åsberg M., A new depression scale designed to be sensitive to change. Br J Psychiatry. 1979; 134:382-389.

Miller I W, et al., The Modified Hamilton Rating Scale for Depression: reliability and validity. Psychiatry Res. 1985;14(2):131-142.

Fava M., Diagnosis and definition of treatment-resistant depression. Biol Psychiatry. 2003; 53(8):649-659.

While various specific embodiments have been illustrated and described, some are represented below. It will be appreciated that various changes can be made without departing from the spirit and scope of the inventive concept(s).

1. A method of treating major depressive disorder, the method comprising administering cariprazine or a pharmaceutically acceptable salt thereof in an amount of 1.5 mg/day or 3 mg/day to a patient in need of adjunctive therapy for major depressive disorder (MDD), wherein the administration of cariprazine achieves statistically significant change from baseline to Week 6 in the Montgomery Åsberg Depression Rating Scale (MADRS) total score.

2. The method of claim 1, wherein the patient in need of adjunctive therapy for MDD meets Diagnostic and Statistical Manual of Mental Disorders, Fifth Edition (DSM-5) criteria for MDD based on Structured Clinical Interview for DSM-5 (SCID-5) and has a total score ≥22 on the Hamilton Depression Rating Scale-17 (HAMD-17) prior to treatment.

3. The method of claim 2, wherein the patient has a major depressive episode of at least 8 weeks to less than 24 months.

4. The method of claim 2, wherein the patient demonstrates inadequate response to 1 to 3 antidepressants of adequate dose and adequate duration as measured by Antidepressant Treatment Response Questionnaire (ATRQ).

5. The method of claim 1, wherein the method of treating major depressive disorder comprises administering cariprazine or a pharmaceutically acceptable salt thereof in an amount of 1.5 mg/day as an adjunct to antidepressant therapy to a patient in need of treatment for major depressive disorder for 6 weeks.

6. The method of claim 5, wherein administration of cariprazine or a pharmaceutically acceptable salt results in a statistically significant remission of the depressive episode as defined by less than 10 total score on MADRS total score.

7. The method of claim 6, the statistically significant remission is achieved with less than 2% to no adverse events leading to discontinuation of therapy.

8. The method of claim 7, wherein cariprazine or a pharmaceutically acceptable salt is cariprazine hydrochloride.

9. The method of claim 1, wherein the method of treating major depressive disorder comprises administering cariprazine or a pharmaceutically acceptable salt thereof in an amount of 3.0 mg/day as an adjunct to antidepressant therapy to a patient in need of treatment for major depressive disorder for 6 weeks.

10. The method of claim 9, wherein administration of cariprazine or a pharmaceutically acceptable salt results in a statistically significant remission of the depressive episode as defined by less than 10 total score on MADRS total score.

11. The method of claim 10, the statistically significant remission is achieved with less than 2% to no adverse events leading to discontinuation of therapy.

12. The method of claim 11, wherein cariprazine or a pharmaceutically acceptable salt is cariprazine hydrochloride.

13. A method of statistically significant treatment of major depressive disorder in patients who had inadequate responses to one or more antidepressants, comprising administering to each said patient cariprazine or a pharmaceutically acceptable salt thereof in an amount of 1.5 mg per day as an adjunctive therapy to one or more said antidepressants, and said treatment achieves statistically significant least square mean change in the MADRS total score in said patients from baseline to week 6 of said treatment as compared to placebo.

14. The method of claim 13, wherein said statistically significant least square mean change in the MADRS total score in said patients from baseline to week 6 of said treatment as compared to placebo is −2 points or exceeds said 2-points.
